(19) United States
(12) Patent Application Publication
(10) Pub. No.: US 2025/0264452 A1
(43) Pub. Date: Aug. 21, 2025

(54) A SYSTEM FOR IN-VITRO DETERMINATION OF A PARAMETER OF A SAMPLE

(71) Applicant: University of Galway, Galway (IE)

(72) Inventors: Ananya Gupta, Galway (IE); O'Halloran Martin, Galway (IE); Sanjeev Gupta, Galway (IE)

(73) Assignee: University of Galway, Galway (IE)

(21) Appl. No.: 18/838,882

(22) PCT Filed: Feb. 20, 2023
(86) PCT No.: PCT/EP2023/054234

§ 371 Date: Apr. 17, 2025

(30) Foreign Application Priority Data
Feb. 18, 2022 (EP) 22157620.0

(51) Int. Cl. G01N 33/49; G01N 15/01; G01N 15/10; G01N 15/1031; G01N 27/02; G01N 33/487

(52) U.S. Cl. CPC G01N 33/49; G01N 15/01; G01N 15/1023; G01N 15/1031; G01N 27/026; G01N 33/48785; G01N 2015/016; G01N 2015/1006; G01N 2015/1024

(57) ABSTRACT

A system (1) for in-vitro determination of a parameter of a whole blood sample, comprising a cartridge (2) comprising an analysis chamber (45) for receipt of a whole blood sample and an impedance microsensor (8) comprising an arrangement of electrodes integrated into the analysis chamber, and a reader device (3) configured for detachable coupling with the cartridge. The reader device comprising a radio frequency (RF) transmitter (20) configured to electrically couple with the impedance microsensor when the reader device is coupled to the cartridge, generate an input RF signal, and transmit the input RF signal to an RF input of the impedance microsensor and a radio frequency (RF) receiver (21) configured to electrically couple with the impedance microsensor when the reader device is coupled to the cartridge to receive an output RF signal from an RF output of the impedance microsensor. The reader device (3) also comprises a computing device (11) electrically coupled to the RF receiver and configured to receive the output RF signal, determine a complex impedance value of the blood sample based on the output RF signal, and calculate the parameter of a blood sample based on the calculated complex impedance value.

### FIELD OF THE INVENTION

The present invention relates to a system for in-vitro determination of a parameter of a sample. In particular, the invention relates to a method for in-vitro determination of a neutrophil count of a blood sample, especially a sample of whole blood.

### BACKGROUND TO THE INVENTION

Neutropenia is a condition of having abnormally low level of neutrophils, a type of white blood cell, required to fight infections, particularly those caused by bacteria. Patients having <1,500 neutrophils per microliter of blood are considered to be Neutropenic. Chemotherapy-induced neutropenia (CIN) occurs in cancer patients as an adverse side effect of cancer treatment. The immune system of these patients becomes greatly impaired making them susceptible to infections, a condition known as Febrile Neutropenia (FN) associated with high body temperature. FN is considered an oncologic emergency, with 60,000 cancer patients in the US hospitalized each year and a mortality rate of 21%.

The current system of management of febrile neutropenia involves administration of prophylactic antimicrobials and hematopoietic growth factor supplements (granulocyte colony stimulating factor G-CSF). However, neutropenia is detected in these patients only after the onset of infection limiting the efficacy of these therapeutic interventions. Timely detection of neutrophil nadir and prevention of systemic infection will improve the outcome of cancer treatment.

Existing blood analysis devices are based on various technologies including “cell counting” where individual cells are isolated within a chamber, classified based on impedance or light scattering characteristics, and then counted within the correct category (red/while/platelet blood cells). These systems are technically complex, costly and difficult to miniaturize, and are therefore unsuitable for home-use.

It is an objective of the invention to overcome at least one of the above-referenced problems.

### SUMMARY OF THE INVENTION

The objective is met by the provision of a device for determining a parameter of a body sample such as blood, for example a cell count of a specific cell type such as neutrophils, red blood cells or platelets, that can be performed using only a small volume of blood. The device employs impedance spectroscopy of the sample to quantify a specific cell type in a sample, and is provided in two-parts, a cartridge comprising a sample analysis chamber and a dielectric microsensor (in particular an impedance sensor), and a separate reader for the cartridge that includes the hardware to generate and transmit a RF signal to the dielectric microsensor and receive an output RF signal from the dielectric microsensor, and software to determine an impedance parameter (e.g. complex impedance) from the output RF signal and calculate a parameter of the sample from the impedance parameter. The cartridge and reader are configured to detachably couple together allowing the reader to be re-used and the cartridge to be discarded after use, thus avoiding cross-contamination issues. The device will enable patients to monitor their own cell counts and allow early detection of a pathology such as neutropenia without the need to present to a clinic or hospital.

In a first aspect, the disclosure provides a system for in-vitro determination of a parameter (e.g. a specific cell count) of a sample (e.g. whole blood or a faction thereof), comprising
a cartridge comprising an analysis chamber for receipt of a sample and an impedance microsensor comprising an arrangement of electrodes integrated into the analysis chamber; and
a reader device configured for detachable coupling with the cartridge, the reader device comprising:
a radio frequency (RF) transmitter configured to electrically couple with the impedance microsensor when the reader device is coupled to the cartridge, generate an input RF signal, and transmit the input RF signal to an RF input of the impedance microsensor;
a radio frequency (RF) receiver configured to electrically couple with the impedance microsensor when the reader device is coupled to the cartridge to receive an output RF signal from an RF output of the impedance microsensor; and
a computing device electrically coupled to the RF receiver and configured to receive the output RF signal, determine a complex impedance value of the blood sample based on the output RF signal, and calculate the parameter of a blood sample based on the calculated complex impedance value.

In any embodiment, the sample is a whole blood sample, or a blood fraction such as plasma or serum or any other fraction.

In any embodiment, the reader device comprises a graphical display operatively coupled to the computing device to display the calculated parameter of the blood sample.

In any embodiment, the computing device is configured to calculate a neutrophil count of the whole blood sample based on the determined complex impedance values of the whole blood sample.

In any embodiment, the impedance microsensor comprises an interdigitated electrode.

In any embodiment, the interdigitated electrode comprises an auxiliary electrode, a reference electrode and first and second interdigitated working electrodes.

In any embodiment, the first interdigitated working electrodes is electrically coupled to the RF input and the second interdigitated working electrodes is electrically coupled to the RF output.

In any embodiment, the cartridge comprises:
a housing in which the analysis chamber is disposed within the housing;
a sample inlet disposed on an external surface of the housing; and
a sample conduit providing fluidic communication between the sample inlet and the analysis chamber.

In any embodiment, the external surface of the housing comprises a lancet disposed adjacent to the sample inlet.

In any embodiment, the sample conduit is selected from a capillary conduit and a microfluidic conduit.

In any embodiment, the housing comprises an overflow chamber fluidically connected to a distal side of the analysis chamber and a viewing window configured to allow a user visually detect the presence of whole blood in the overflow chamber.

In any embodiment, the housing comprises a venting conduit to vent air from the analysis chamber.

In any embodiment, the viewing window and venting conduit are disposed on a top of the housing.

In any embodiment, the housing has a proximal end comprising the sample inlet and a distal end comprising the RF input and RF output.

In any embodiment, the cartridge comprises a plurality of sample inlets and associated sample conduits.

In any embodiment, the system comprises a detachable cover for the or each sample inlet.

In any embodiment, the dielectric microsensor is disposed on a base of the analysis chamber.

In any embodiment, the analysis chamber has a volume of 1 to 50 μl.

In any embodiment, the interdigitated working electrodes comprise a band gap of 5 to 50 μm.

In any embodiment, the interdigitated working electrodes comprise a band width of 5 to 50 μm.

In any embodiment, a surface area of the interdigitated working electrodes is 1 mm² to 20 mm².

In any embodiment, the reader device comprises a socket dimensioned to receive part of the cartridge including the RF input and RF output.

In any embodiment, the cartridge and reader device are configured to magnetically couple together.

In any embodiment, the RF transmitter is configured to generate a resonant RF input signal

In any embodiment, the RF transmitter is configured to generate a RF input signal with a frequency range of 0.5 to 50 KHz.

In any embodiment, the RF transmitter is configured to generate a RF input signal with a frequency range of 0.5 to less than 20 KHz.

In any embodiment, the RF transmitter is configured to generate a RF input signal with a frequency range of 0.5 to 1.5 KHz.

In any embodiment, the reader device comprises a wireless transmitter operably coupled to the computing device and configured for near-field transmission of an output of the computing device to a local device.

In any embodiment, the computing device is configured to calculate a parameter of the whole blood sample based on a plurality of measured complex impedance values of the blood sample measured by the impedance microsensor over a time period.

In any embodiment, the computing device comprises:
an algorithm trained using complex impedance training data obtained from blood samples from a training set of subjects and corresponding known blood parameter data; and
a processor configured to:
   receive a determined complex impedance value for a blood sample from a test subject; and
   apply the algorithm to the determined complex impedance value so as to determine the blood parameter of the blood sample from the test subject.

In any embodiment, the algorithm is a signal classifier algorithm. In any embodiment, the signal classifier algorithm is a Support Vector Machine (SVM) machine learning algorithm. In any embodiment, the signal classifier algorithm is a Linear Discriminant Analysis (LDA) algorithm.

In another aspect, the invention provides cartridge for in-vitro determination of a parameter of a sample, comprising:
an analysis chamber for receipt of a sample; and
an impedance microsensor comprising an arrangement of electrodes integrated into the analysis chamber.

In any embodiment, the impedance microsensor comprises a RF input configured to electrically couple the arrangement of electrodes with a RF transmitter and a RF output configured to electrically couple the arrangement of electrodes with a RF receiver.

In any embodiment, the sample is a whole blood sample, or a blood fraction such as plasma or serum or any other fraction.

In any embodiment, the impedance microsensor comprises an interdigitated electrode.

In any embodiment, the interdigitated electrode comprises an auxiliary electrode, a reference electrode and first and second interdigitated working electrodes.

In any embodiment, the first interdigitated working electrodes is electrically coupled to the RF input and the second interdigitated working electrodes is electrically coupled to the RF output.

In any embodiment, the cartridge comprises:
a housing in which the analysis chamber is disposed within the housing;
a sample inlet disposed on an external surface of the housing; and
a sample conduit providing fluidic communication between the sample inlet and the analysis chamber.

In any embodiment, the external surface of the housing comprises a lancet disposed adjacent to the sample inlet.

In any embodiment, the sample conduit is selected from a capillary conduit and a microfluidic conduit.

In any embodiment, the housing comprises an overflow chamber fluidically connected to a distal side of the analysis chamber and a viewing window configured to allow a user visually detect the presence of whole blood in the overflow chamber.

In any embodiment, the housing comprises a venting conduit to vent air from the analysis chamber.

In any embodiment, the viewing window and venting conduit are disposed on a top of the housing.

In any embodiment, the housing has a proximal end comprising the sample inlet and a distal end comprising the RF input and RF output.

In any embodiment, the cartridge comprises a plurality of sample inlets and associated sample conduits.

In any embodiment, the cartridge comprises a detachable cover for the or each sample inlet.

In any embodiment, the impedance microsensor is disposed on a base of the analysis chamber.

In any embodiment, the analysis chamber has a volume of 1 to 50 μl.

In any embodiment, the interdigitated working electrodes comprise a band gap of 5 to 50 μm.

In any embodiment, the interdigitated working electrodes comprise a band width of 5 to 50 μm.

In any embodiment, a surface area of the interdigitated working electrodes is 1 mm² to 20 mm².

In any embodiment, the cartridge is configured to magnetically couple to a reader device

In another aspect, the disclosure provides a reader device configured for detachable coupling with a cartridge of the type comprising a sample analysis chamber and an impedance microsensor, the reader device comprising:
a radio frequency (RF) transmitter configured to electrically couple with the impedance microsensor when the reader device is coupled to the cartridge, generate an input RF signal, and transmit the input RF signal to an RF input of the impedance microsensor;
a radio frequency (RF) receiver configured to electrically couple with the impedance microsensor when the reader device is coupled to the cartridge to receive an output RF signal from an RF output of the impedance microsensor; and
a computing device electrically coupled to the RF receiver and configured to receive the output RF signal, determine a complex impedance value of the sample based on the output RF signal, and calculate the parameter of a sample based on the calculated complex impedance value.

In any embodiment, the sample is a whole blood sample, or a blood fraction such as plasma or serum or any other fraction.

In any embodiment, the reader device comprises a graphical display operatively coupled to the computing device to display the calculated parameter of the blood sample.

In any embodiment, the reader device comprises a socket dimensioned to receive part of the cartridge including an RF input and RF output of a cartridge.

In any embodiment, the reader device is configured to magnetically couple with a reader device

In any embodiment, the RF transmitter is configured to generate a resonant RF input signal

In any embodiment, the RF transmitter is configured to generate a resonant RF input signal with a frequency range of 0.5 to 1.5 KHz.

In any embodiment, the reader device comprises a wireless transmitter operably coupled to the computing device and configured for near-field transmission of an output of the computing device to a local device.

In any embodiment, the computing device is configured to calculate a parameter of the whole blood sample based on a plurality of measured impedance values of the blood sample measured by the dielectric microsensor over a time period.

In any embodiment, the computing device comprises:
an algorithm trained using impedance (e.g. complex impedance) training data obtained from blood samples from a training set of subjects and corresponding known blood parameter data; and
a processor configured to:
   receive a determined impedance value for a blood sample from a test subject; and
   apply the algorithm to the determined impedance value so as to determine the blood parameter of the blood sample from the test subject.

In any embodiment, the algorithm is a signal classifier algorithm, for example a Support Vector Machine (SVM) machine learning algorithm.

In another aspect, the disclosure provides a method of determining a parameter of a blood sample employing the system of the invention.

Typically, the method comprising the steps of adding a sample (e.g. a whole blood sample or a fraction thereof) to the analysis chamber of the cartridge;
coupling the cartridge to the reader device;
actuating the RF transmitter to provide an input RF signal to the dielectric microsensor;
receiving by the RF receiver an output RF signal from the dielectric microsensor in response to the input RF signal;
determining by the computing device an impedance value of the sample based on the output RF signal;
analysing by the computing device the impedance value of the sample to calculate the parameter of the sample;
optionally, displaying the parameter of the sample on the graphical display of the reader device; and
detaching the cartridge form the reader device.

In any embodiment, the method is a method of determining a neutrophil count of the sample.

In any embodiment, the input radio frequency (RF) signal is a resonant RF signal.

In any embodiment, the input radio frequency (RF) signal is a RF signal. in a frequency range of 0.5 to 50 Hz.

In any embodiment, the input radio frequency (RF) signal is a RF signal. in a frequency range of 0.5 to less than 20 KHz.

In any embodiment, the input radio frequency (RF) signal is a RF signal. in a frequency range of 0.5 to 1.5 Hz.

In any embodiment, the method comprises a step of employing a lancet disposed on the cartridge to pierce the skin of a patient.

In another aspect, the invention provides a method of determining a neutrophil count of a sample (e.g. a whole blood sample or a fraction thereof) from a test subject using an impedance (e.g. complex impedance) value of the sample, the method comprising the steps:
obtaining impedance (e.g. complex impedance) training data using samples from a training set of subjects and known neutrophil counts corresponding to the impedance training data;
training an algorithm using the impedance training data and corresponding known neutrophil counts;
obtaining an impedance value (e.g. complex impedance value) for a sample from a test subject; and
applying the trained algorithm to the impedance value of the sample from the test subject so as to determine a neutrophil count for the sample of the test subject.

Typically, the impedance data or value is complex impedance data or a complex impedance value.

In any embodiment, the sample is a blood sample or a fraction thereof. In any embodiment, the blood sample is a whole blood sample.

In any embodiment, the trained algorithm is a signal classifier algorithm. In any embodiment, the signal classifier algorithm is a Support Vector Machine (SVM) machine learning algorithm.

In any embodiment, the complex impedance value of the sample is determined with an impedancemicrosensor, typically comprising an interdigitated electrode.

In another aspect, there is provided computer program product comprising a computer usable medium, where the computer usable medium comprises a computer program code that, when executed by a computer apparatus, determines a parameter (e.g. a count of a specific cell type) of a sample (e.g. a biological sample such as whole blood or a fraction thereof) employing the system of the invention.

Other aspects and preferred embodiments of the invention are defined and described in the other claims set out below.

### BRIEF DESCRIPTION OF THE FIGURES

FIG. 1 is a block diagram illustrating a system of the invention comprising a sample analysis cartridge and a reader device that in this embodiment includes a graphical display and a wireless communication module configured to communicate with a separate device by near-field wireless communication protocol.

FIG. 2A is a perspective view of one embodiment of a cartridge of the invention illustrating the sample analysis chamber and interdigitated electrode (IDE).

FIG. 2B is an exploded view of the cartridge of FIG. 2A.

FIG. 2C is a top plan view of the cartridge of FIG. 2C showing the air vent aperture and overflow chamber viewing aperture.

FIG. 2D is a sectional top plan view of the cartridge of FIG. 2C showing the analysis chamber, overflow chamber, conduit and IDE.

FIGS. 3A to 3D illustrate the use of the system of the invention in which:

FIG. 3A illustrates the use of the lancet to prick a subject finger and draw blood;

FIG. 3B illustrates a blood droplet from the pricked finger being transmitted into the conduit of the cartridge via the sample inlet;

FIG. 3C illustrates how a user can determine when the blood sample has filled the analysis chamber by viewing spill-over blood in the overflow chamber via the viewing winder (pin hole aperture); and

FIG. 3D illustrates the cartridge about to be mounted in a socket of a reader device.

FIG. 4A is a perspective view of another embodiment of a cartridge of the invention having a blood receptacle stage and blood inlet extending from a proximal end of the cartridge.

FIG. 4B is an exploded view of the cartridge of FIG. 4A.

FIG. 4C is a sectional top plan view of the cartridge of FIG. 4A showing the blood receptacle stage, sample analysis chamber and interdigitated electrode (IDE).

FIG. 5A is a perspective view of another embodiment of a cartridge of the invention having a blood receptacle stage and blood inlet disposed on a top surface of the cartridge.

FIG. 5B is an exploded view of the cartridge of FIG. 5A.

FIG. 5C is a top plan view of the cartridge of FIG. 5A showing the air vent aperture and overflow chamber viewing aperture.

FIG. 5D is a sectional top plan view of the cartridge of FIG. 5A showing the analysis chamber, overflow chamber, conduit and IDE.

FIG. 6 illustrates three types of interdigitated electrodes (IDE's), a finger IDE (FIG. 6A), a spiral IDE (FIG. 6B), and a square IDE (FIG. 6C).

FIG. 7A illustrates a finger IDE in more detail.

FIG. 7B illustrates the interdigitated comb-like section of the working electrodes of the IDE of FIG. 5A.

FIGS. 8A and 8B: Relationship between complex impedance of the sample to the neutrophil count at KHz frequency.

FIG. 9: A visualization of the optimized classifier, the training data, as well as the prediction probabilities.

FIG. 10A illustrates a reader device with sensor interface comprising four conducting wires which re electrically coupled to the PCB board.

FIG. 10B illustrates the reader device with the IDE inserted into the sensor interface and the four electrodes of the IDE contacting the four wires of the sensor interface

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “cartridge” or “impedance spectroscopy microsensor device” (which are used herein interchangeably) refer to a device comprising a sample analysis chamber and an impedance microsensor disposed within the analysis chamber. The analysis chamber is dimensioned so that it can be filled with one or a few drops of the sample. The impedance microsensor is generally disposed at the base of the analysis chamber to ensure that it is covered with the sample when the sample is added to the chamber. The chamber comprises a sample inlet disposed on an external surface of the cartridge (for example an end of the cartridge or a top surface of the cartridge), and a conduit providing fluidic communication between the sample inlet and the analysis chamber. When the sample inlet is provided on a top surface of the cartridge the inlet may be funnel shaped and may feed directly into the analysis chamber. The conduit may be a microfluidic conduit or a capilliary conduit. The cartridge comprises a housing containing the analysis chamber. The housing may be elongated and have a distal end and a proximal end. The sample inlet may be provided at a distal end. The analysis chamber generally has a rectangular footprint, but may have a different shape footprint, for example square, round or oval. The analysis chamber may be dimensioned such that 30 μl of blood will cover the base of the chamber. The analysis chamber may have a volume of 1 to 50 μl, preferably 20 to 50 μl. The analysis chamber may contain an overflow chamber which may be disposed on a distal side of the analysis chamber (for example, on an opposite end of the analysis chamber to the fluid inlet conduit). The cartridge may be 20 to 120 mm in length and 5 to 30 mm in width. The cartridge may comprise an air vent for the analysis chamber, for example a small air vent that exits on a top surface of the housing. The cartridge may also include one, two or more viewing windows for the overflow chamber to allow a user determine when the blood has entered the overflow chamber.

As used herein, the term “reader device” refers to a device configured to detachably couple with the cartridge. The reader device incudes a RF signal transmitter and a RF signal receiver. The RF signal transmitter generally comprises a RF signal generator. The reader device is configured to electrically couple with the impedance microsensor when the reader is coupled to the cartridge to send input RF signals to the impedance microsensor and receive output RF signals from the impedance microsensor. The reader device also includes a computing device configured to determine an impedance parameter such as complex impedance from the output RF signal and typically calculate a parameter of the sample from the determined impedance parameter. The parameter may be a parameter of a type of cell in the sample, for example a relative or absolute determination of cell numbers for a specific cell type. Examples of cells that can be enumerated with the device and system of the invention include neutrophils, platelets and red blood cells. Enumeration of neutrophils is useful for monitoring for neutropenia and for general health, particularly in patients with specific diseases or conditions or undergoing specific treatments which can affect the immune system. The software may comprise an algorithm trained using impedance training data obtained from samples from a training set of subjects and corresponding known sample parameter data. The software may comprise a processor configured to receive a determined complex impedance value for the sample from a test subject and apply the algorithm to the determined complex impedance value so as to determine the parameter of the sample from the test subject. The algorithm may be a signal classifier algorithm such as a Support Vector Machine (SVM) machine learning algorithm.

As used herein the term “sample” refers to any sample such as a biological, environmental or industrial sample. The sample is generally a liquid sample. The invention is especially directed for use with biological sample, especially blood samples or fractions thereof such as plasma and serum and in particular whole blood samples. Other biological samples that may be employed include urine, saliva, sweat, cerebrospinal fluid, semen, and synovial fluid.

As used herein, the term “impedance microsensor” refers to a sensor configured to detect an impedance parameter of a sample by electric impedance spectroscopy, for example complex impedance. The sensor is miniaturised so as to fit within an analysis chamber of a sample analysis cartridge. In one embodiment, the impedance microsensor is a screen printed electrode. Screen printed microelectrodes are described in Torre et al (Biosensors 2020, 10, 139), Mishra et al. (Encyclopedia of Interfacial Chemistry; Elsevier: Amsterdam, The Netherlands, 2018; pp. 487-498), Smart et al. (Trends Anal. Chem. 2020, 127, 115898), and Vasilescu et al. (Sensors 2016, 16, 1863). In one embodiment, the impedance microsensor is a screen printed interdigitated electrode.

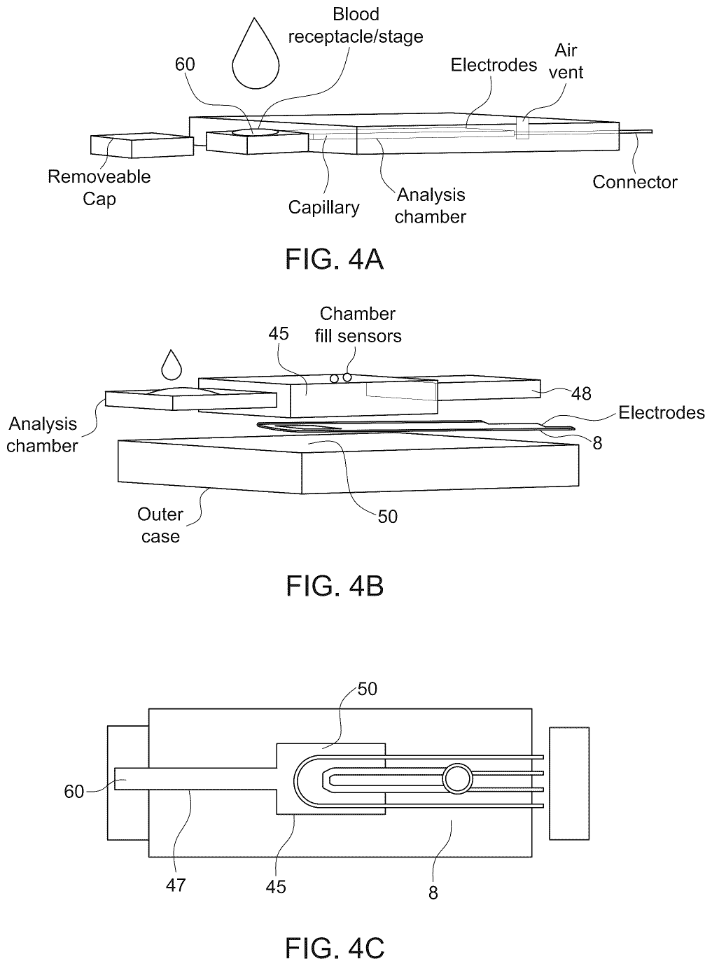

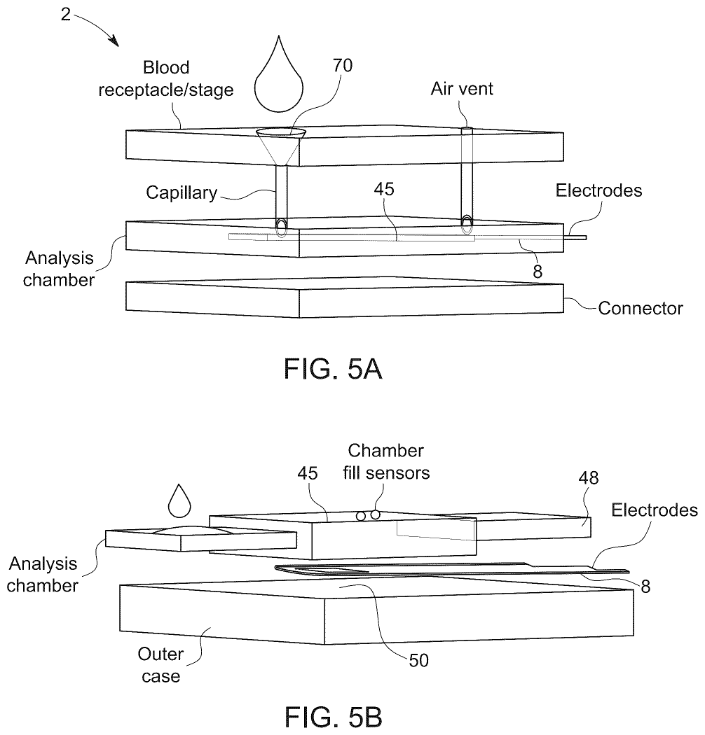

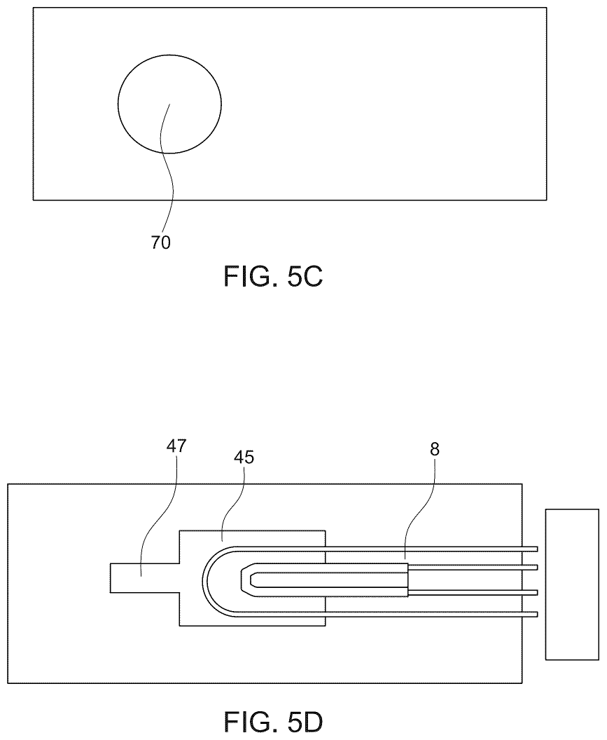

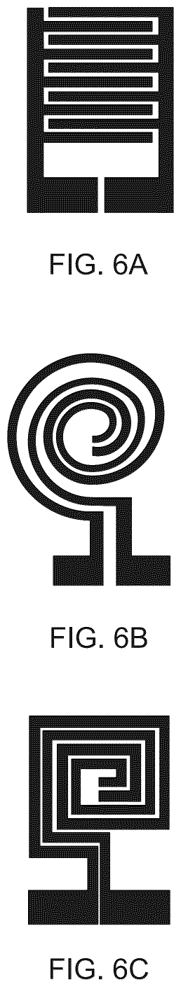

As used herein, the term “interdigitated electrode” or “IDE” refers to an impedance microsensor comprising two individually addressable interdigitated comb-like electrode structures. In order to detect and analyze a biochemical molecule or analyte, the impedance and capacitance signal need to be obtained. The impedance biosensor depends on the resistance and capacitance while the capacitance biosensor influenced by the dielectric permittivity. However, the geometry and structures of the interdigitated electrodes affect both impedance and capacitance biosensor. The IDE may be of the finger, spiral or square format (FIG. 4). In one embodiment, the IDE comprises two working electrodes with an auxiliary electrode and a reference electrode (FIG. 5). The electrodes may be manufactured in the same material, and are generally provided on a substrate, typically by optical lithography technology. The substrate may be glass, but other substrates are envisaged. Typically, the IDE is a screen printed IDE. One of the working electrodes is generally configured to receive an input RF signal and one is generally configured to emit an output RF signal. The auxiliary electrode is generally used to make a connection to the electrolyte for the purpose of applying a current to the working electrode. The material used to make an auxiliary electrode is typically an inert material like graphite or a noble metal such as gold, carbon or platinum. The purpose of the reference electrode is generally to provide a stable potential for controlled regulation of the working electrode potential and in doing so allow the measurement of the potential at the working electrode without passing current through it. The reader generally analyses the output RF signal and determines an impedance parameter such as complex impedance of the output RF signal, from which it calculates the parameter of the sample under investigation.

Interdigitated electrodes as impedance and capacitance electrodes are described in Mazlan et al. (AIP Conference Proceedings 1885, 020276 (2017)) and are commercially available from METROHM. In any embodiment, the interdigitated working electrodes comprise a band gap of 5 to 50 μm, 5 to 30 μm, 5 to 20 μm, or 5 to 15 μm. In any embodiment, the interdigitated working electrodes comprise a band width of 5 to 50 μm, 5 to 30 μm, 5 to 20 μm, or 5 to 15 μm. In any embodiment, a surface area of the interdigitated working electrodes is 1 to 20 mm², 1 to 10 mm², 1 to 5 mm² or 3 to 5 mm².

The sample that is analysed may be untreated. As used herein, the term “untreated” as applied to the sample (e.g the whole blood sample) should be understood to mean that the sample is not treated to lyse cells in the blood and not treated with a binding agent configured to bind with one or more cell types in the sample, prior to or during analysis. Thus, in any embodiment, the invention relates to a system and method to determine a parameter, such as a cell count of a specific cell type in a sample, that employs a sample in which the cells in the sample do not need to be pretreated to either lyse one or more types of cells or to attach a binding agent to the cells in the sample. In any embodiment the method of the invention does not employ cell counting using a cell sizing principle. In an embodiment the system of the invention does not comprise a Coulter chamber.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

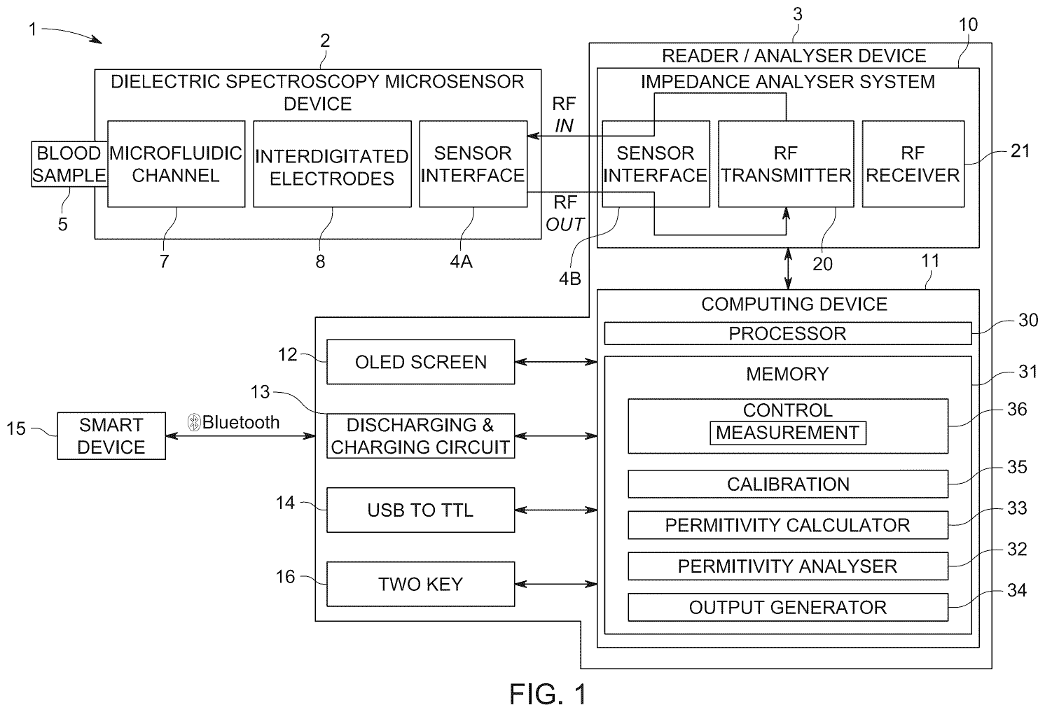

Referring to the drawings and initially to FIG. 1, a system of the invention is illustrated in block diagram format. In this embodiment, the system is for counting neutrophils in a whole blood sample, although it will be appreciated that the system may be employed to count different cell types in different types of samples.

The system comprises a cartridge (impedance spectroscopy microsensor device) 2 and a separate reader/analyser device 3. The cartridge and reader device are configured to detachably couple together, and each of the cartridge and reader have a sensor interface 4A, 4B that are configured to electrically couple together when the reader 3 and cartridge 2 detachably connect together.

The cartridge device 2 comprises a housing with a sample inlet 5, sample analysis chamber 6, and a microfluidic conduit 7 providing fluidic communication between the analysis chamber and the sample inlet. An interdigitated electrode (IDE) 8 is disposed in the analysis chamber and the electrodes of the IDE are in electrical communication with the sensor interface 4A.

The reader/analysis device 3 comprises an impedance analyser module 10 and a computing device module 11. It also includes a graphical display (OLED screen) 12, a discharging and charging circuit 13, a USB port 14, a Bluetooth wireless communication module 15, and a two key 16.

The impedance analyser module 10 comprises a RF signal generator/transmitter 20, a RF receiver 21. The RF transmitter is electrically coupled to the sensor interface 4B to transmit an input RF signal to the IDE 8 via the respective sensor interfaces. The RF receiver 21 is electrically coupled to the sensor interface to receive an output RF signal from the IDE via the respective sensor interfaces.

The computing device module 11 comprises a processor module 30 and a memory module 31. The memory module comprises an impedance analyser module 32, an impedance calculator module 33, an output generator 34, a calibration module 35 and a control/measurement module 36. The computing device is configured to receive the output RF signal from the RF receiver 21, analyse the output RF signal and determine an impedance value for the output RF signal, and then calculate a neutrophil count for the whole blood sample based on the determined impedance value.

The system is configured to determine a neutrophil count for a sample of whole blood, display the neutrophil count of the screen of the reader device along with additional data, for example whether the count is low, normal or high. The system may also relay the data to a local device such as a smart phone, tablet or computer. The local device may comprise software configured to allow the local device communicate with the reader device, and relay the data by the local device to a remote location, for example to a physician, a hospital or clinic, or an emergency service. The system is suitable for at-home use without the need for any specialised equipment or expertise.

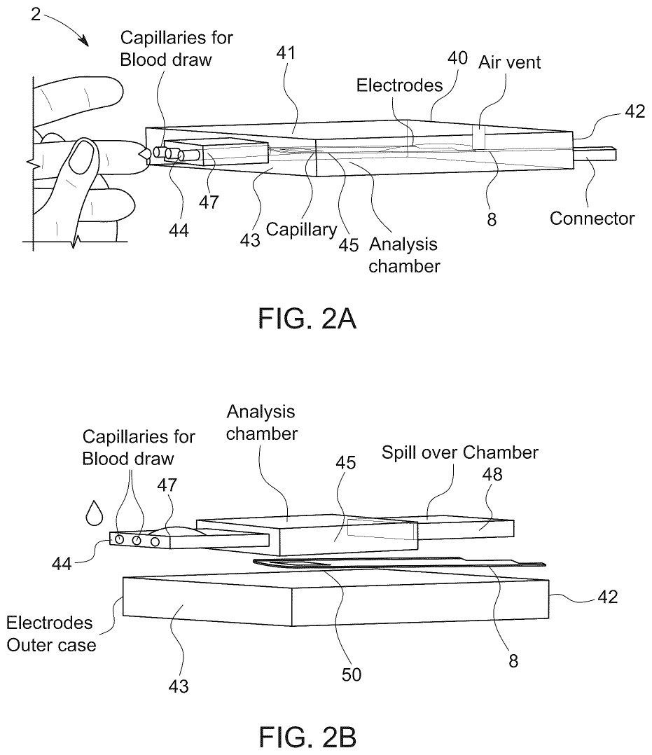

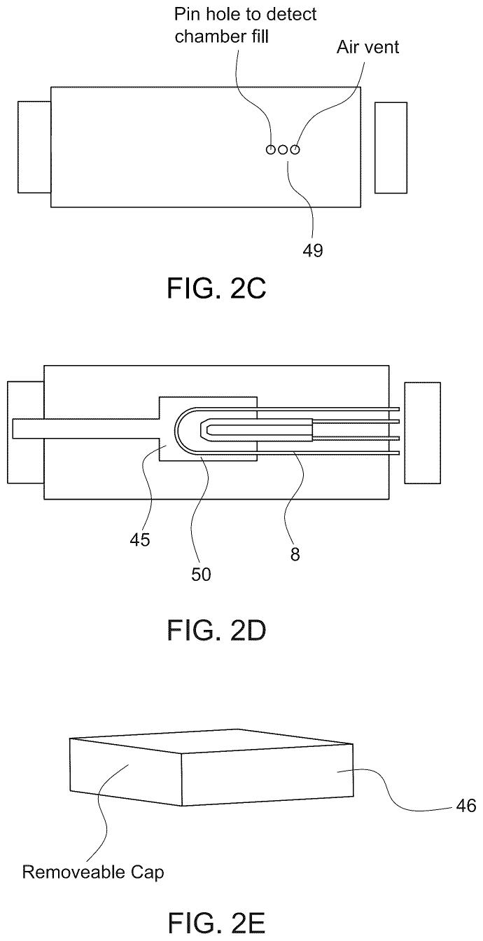

Referring to FIGS. 2A to 2E, a cartridge 2 according to the invention is illustrated and described. The cartridge 2 comprises an elongated housing 40 with a top surface 41, opposed bottom surface (not shown), distal end 42, and proximal end 43. In the embodiment shown the cartridge has a length of about 10 cm and a width of about 3 cm. A sample analysis chamber 45 is provided in the housing towards a proximal end of the device, and three sample inlets 44 provided on an extension at the proximal end 43 provide fluidic communication to the analysis chamber 45 via microfluidic conduits 47. A sample overflow (spill-over) chamber 48 is provided on a distal side of the analysis chamber. Pin holes 49 are provided on the top surface 41, two of which (49A) overlie the overflow chamber, and one of which (49B) provides an air vent for the analysis chamber as illustrated in FIG. 2C. FIG. 2E illustrates a removable cap 46 that can cover the fluid inlets.

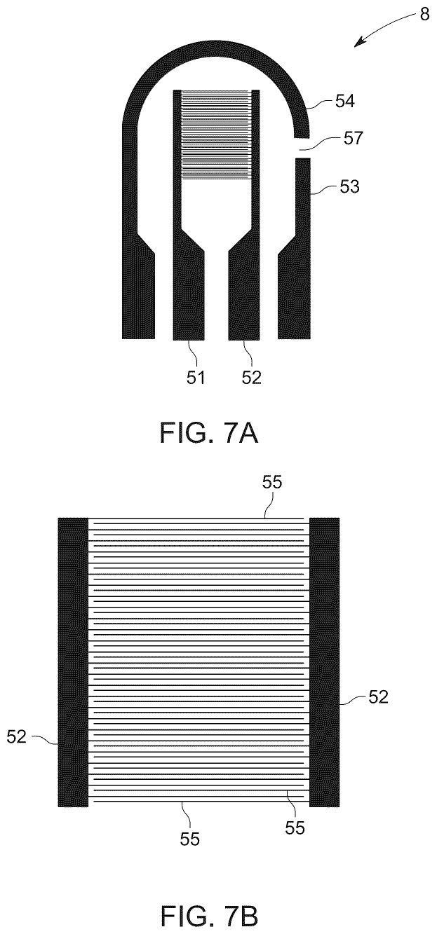

The cartridge 2 comprises a finger-type interdigitated electrode 8 having a sensing end 50 disposed on the base of the analysis chamber 45. As illustrated in more detail in FIGS. 7A and 7B, the finger-type interdigitated electrode 8 comprises a first working electrode 51, second working electrode 52, reference electrode 53 and auxiliary electrode 54. Each of the working electrodes 51 and 52 comprise comb-like electrode structures 55 which are interdigitated as illustrated in FIG. 7B. The interdigitated working electrodes comprise a band gap of 5 to 50 μm, a band width of 5 to 50 μm, and a surface area of about 1 to 20 mm². The reference electrode 53 and the auxiliary electrode 54 terminate in the analysis chamber 45 defining a gap 57 between their respective ends. The electrodes terminate at the distal end 42 of the cartridge with connecting terminals configured to electrically connect with the reader device when the cartridge and reader device are coupled together.

Although not illustrated, the proximal end 43 of the cartridge may include a lancet disposed adjacent the sample inlet. This allows a user prick their finger with the end of the cartridge and load blood into the cartridge with a single device, obviating the need for a separate lancet.

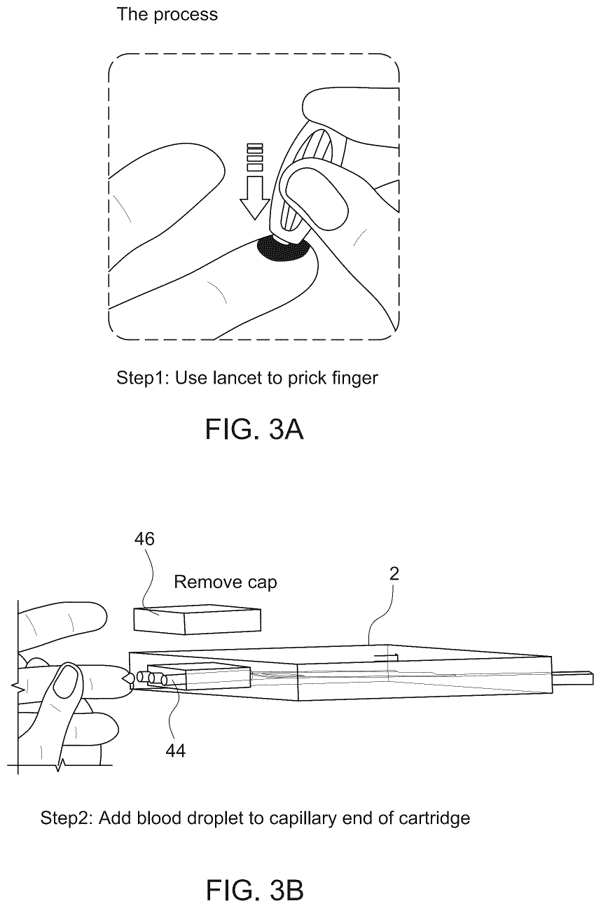

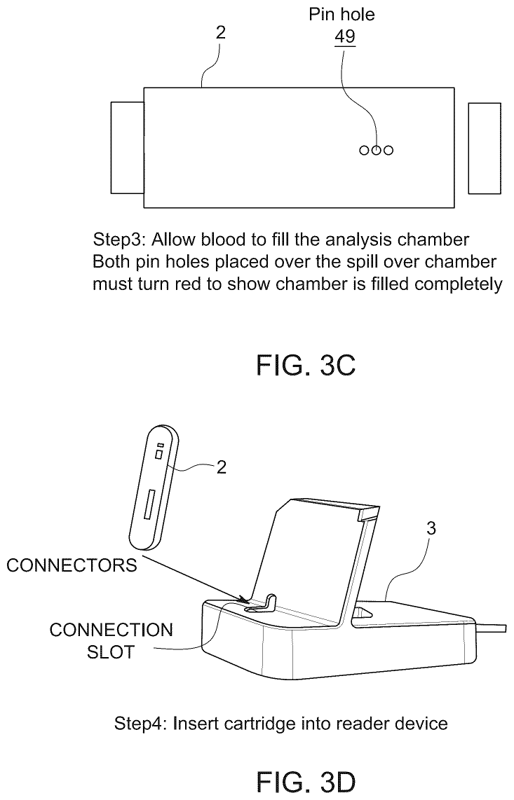

Referring to FIG. 3, the use of the device of the invention to assay a whole blood sample for neutrophil count is described. In a first step (FIG. 3A), a lancet is used to prick a subject's finger, before the sample inlets 44 of the cartridge 2 are brought into contact with a drop of blood (FIG. 3B). The blood enters the sample inlet and travels along the conduit into the analysis chamber by capillary action until the sample chamber is filled and the IDE is submerged under blood. It's possible for a user to determine when the analysis chamber is full as blood will pass into the overflow chamber and can be seen through the two viewing pinholes 49A disposed directly over the overflow chamber which will turn red indicating that the analysis chamber is fully charged with blood (FIG. 3C). The cartridge is then inserted into a socket in the reader device 3, where the IDE electrically couples with the RF transmitter and RF receiver of the impedance analyser module 10 (FIG. 3D). The computing device of the reader/analyser device then analyses the RF output of the IDE and determines an impedance value of the sample, and then calculates a neutrophil count of the blood sample based on the determined impedance of the sample. In this embodiment, the impedance of whole blood was measured at a single resonant frequency of 1 kHz.

Referring to FIGS. 4A to 4C, a cartridge according to an alternative embodiment of the invention is described in which parts described with reference to the previous embodiments are assigned the same reference numerals. In this embodiment, the blood inlet 60 on the extension at the proximal end of the cartridge is disposed on a top of the extension as opposed to the end of the extension allowing a drop of blood to be loaded onto the inlet 60 from above.

Referring to FIGS. 5A to 5D, a cartridge according to an alternative embodiment of the invention is described in which parts described with reference to the previous embodiments are assigned the same reference numerals. In this embodiment, the blood inlet 70 is provided as a funnel-shaped aperture on the top surface of the cartridge that feeds directly into a proximal end of the analysis chamber.

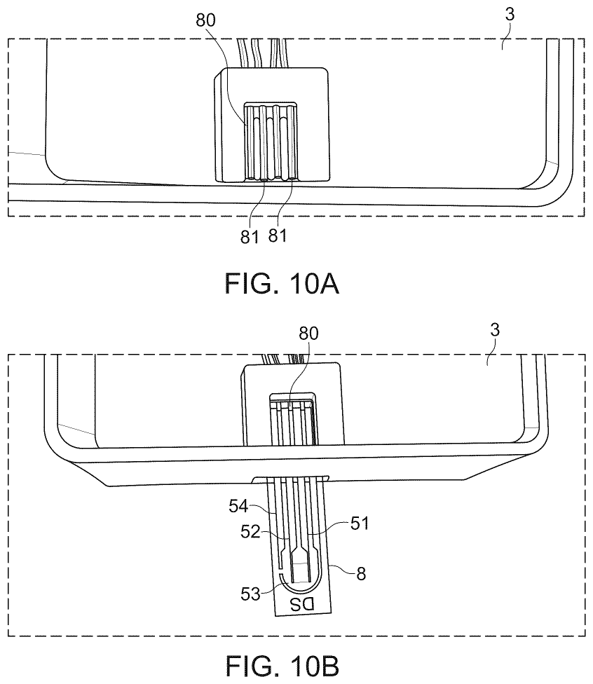

FIG. 10A illustrate the sensor interface of the reader device 3 comprising a socket 80 containing four conducting wires 81 each of which is electrically coupled to a PCB board of the reader device (not shown). FIG. 10B illustrates a distal end of the IDE 8 inserted into the socket 80 with the working, reference and auxiliary electrodes 51, 52, 53 and 54 making electrical contact with the conducting wires 81.

### Calculation of Impedance of Sample

By using the voltage generated between auxiliary electrode and the reference electrode, and the varying current generated between the two working electrodes, the values of the real and imaginary parts of the impedance of the sample at a certain frequency can be calculated.

$$V ( t ) = \overline{V} + \hat{V} \cdot sin ( ω t )$$

$$I ( t ) = \overline{I} + \hat{I} \cdot sin ( ω t + ϕ )$$

$$\begin{matrix}Z ( j ω ) = \frac{V ( j ω )}{I ( j ω )} = \frac{\hat{V}}{\hat{I}} \cdot {e}^{- j ϕ} = \vert Z \vert \cdot {e}^{j \cdot Arg ( Z )} \\ = Re ( Z ) + j \cdot Im ( Z ) .\end{matrix}$$

f is the test signal frequency; ω=2πf, the angular frequency; ϕ the phase difference between V(t) and I(t); V(jω) and I(jω) the Steinmetz transforms of V(t) and I(t), respectively. The sine-wave parameters can be calculated from the acquired signals using a fitting algorithm in the time domain or by applying a fast Fourier transform algorithm.

### Calculation of Neutrophil Count

The system of the invention uses impedance spectroscopy of whole blood to detect changes in neutrophil count. In this embodiment, the complex impedance of whole blood is measured at a single resonant frequency of 1 kHz. As the impedance measured at a given frequency has been discovered to exhibit a strong correlation with the neutrophil count, this allows the impedance of the sample to be correlated to the neutrophil count.

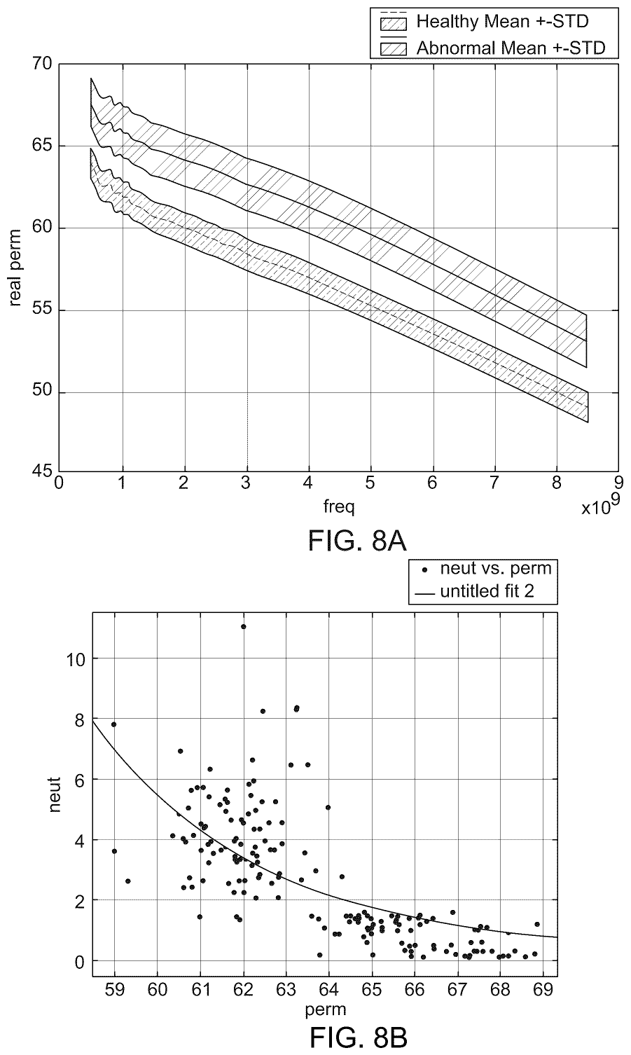

As illustrated in FIGS. 8A and 8B, the permittivity of the blood samples with low neutrophil counts below 1.0 show a good separation from the blood samples with normal neutrophil count of 1.5 or above. The blood samples separate out into two very distinct groups normal and abnormal based on their impedance. So simply by measuring the impedance we can detect of the blood sample has a low neutrophil count. This strong correlation helps us to then use a SVM learning algorithm that can be trained to predict the neutrophil count based on the impedance measurement.

The correlation between the impedance and the neutrophil count is parabolic. The impedance measurement also shows a linear correlation with the hematocrit to RBC count. Therefore, the algorithm can be trained to predict neutrophil counts based on the impedance measured. An algorithm can also be trained and developed to predict changes in RBC or hematocrit. To train the algorithm we compare our predicted neutrophil count to the biochemistry results obtained from hematology labs where cell count is measurement by the hospital gold standard BD flow cytometer cell counter.

The differences in impedance between the two populations (normal and abnormal) highlights the possibility of using a resonant (single frequency) device to calculate Neutrophil concentration based on an impedance characterization of a blood sample. In order to assess the feasibility of such a device, the impedance measurements at a single frequency will be used in conjunction with a machine learning algorithm to create a prediction model that uses impedance measurements to predict the onset of neutropenia.

### Development and use of Support Vector Machine (SVM) Algorithm

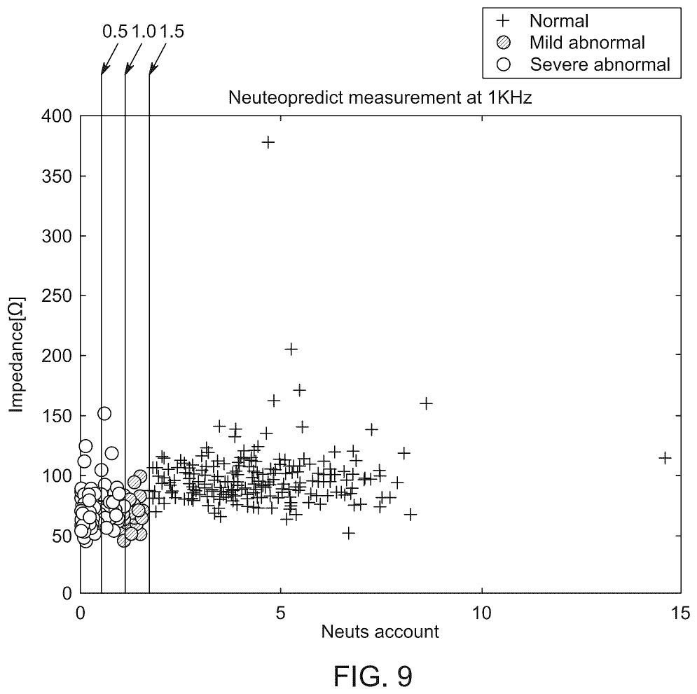

Support vector machines (SVM) is a machine learning algorithm that can be used for binary classification, in this case, whether a blood sample is from a healthy patient or a patient with neutrophil. An SVM-classifier must first be trained in order to predict the outcome classes. The real and imaginary components of the permittivity of whole blood at 1 KHz can be used as the two input features for the SVM-classifier. The training data consisted of 355 blood samples and represented 75% of the complete data set. These 355 training samples are divided into 3 categories based on the number of neutrophils, normal, mild abnormal, severe abnormal and given three labels of 1, 0, and −1. The final SVM model parameters is using radial basis function as the kernel for SVM classifier. The optimal solution to build the model is found by a function that automatically finds the optimal loss parameter. Once these optimized parameters are found they are used to train the final SVM classifier that will be used to predict the classes of the test data as shown in FIG. 9 and Tables 1 to 5 below.

TABLE 1

| Normal | Abnormal | Total |
|---|---|---|
| 235 | 239 | 474 |

TABLE 2

| | |
|---|---|
| Dataset | 474 |
| Training dataset | 355 |
| Testing dataset | 119 |

TABLE 3

| Neutrophil Quantity | Status | Category Label |
|---|---|---|
| >1.5 | Normal | 1 |
| 1.0 to 1.5 | Mild abnormal | 0 |
| <1.0 | Severe abnormal | −1 |

TABLE 4

| Actual | | | |
|---|---|---|---|
| 1 | 58 | | 1 |
| 0 | | 7 | 3 |
| −1 | | | 50 |
| | 1 | 0 | −1 |

Predicted

TABLE 5

| | |
|---|---|
| Dataset | 119 |
| Sensitivity | 98.3% |
| Specificity | 100% |

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A system (1) to enumerate a specific cell type in a whole blood sample, comprising
a cartridge (2) comprising an analysis chamber (45) for receipt of a whole blood sample and an impedance microsensor (8) comprising an arrangement of electrodes integrated into the analysis chamber; and
a reader device (3) configured for detachable coupling with the cartridge, the reader device comprising:
a radio frequency (RF) transmitter (20) configured to electrically couple with the impedance microsensor when the reader device is coupled to the cartridge, generate an input RF signal, and transmit the input RF signal to an RF input of the impedance microsensor (8);
a radio frequency (RF) receiver (21) configured to electrically couple with the impedance microsensor (8) when the reader device is coupled to the cartridge to receive an output RF signal from an RF output of the impedance microsensor; and
a computing device (11) electrically coupled to the RF receiver and configured to receive the output RF signal, determine a complex impedance value of the blood sample based on the output RF signal, and calculate a cell count of the specific cell type in the blood sample based on the calculated complex impedance value.

2. A system according to claim 1, in which the analysis chamber does not comprise a Coulter counter.

3. A system according to claim 1, in which the computing device (11) is configured to calculate a neutrophil count of the whole blood sample based on the determined complex impedance values of the whole blood sample.

4. A system according to claim 1, in which the impedance microsensor (8) comprises an interdigitated electrode.

5. A system according to claim 4, in which the interdigitated electrode comprises an auxiliary electrode (54), a reference electrode (53) and first and second interdigitated working electrodes (51, 52), in which the first interdigitated working electrode (51) is electrically coupled to the RF input and the second interdigitated working electrodes (52) is electrically coupled to the RF output.

6. A system according to claim 1, in which the cartridge (2) comprises:
a housing in which the analysis chamber (45) is disposed within the housing;
a sample inlet (44, 60, 70) disposed on an external surface of the housing; and
a sample conduit (47) providing fluidic communication between the sample inlet and the analysis chamber.

7. A system according to claim 6, in which the external surface of the housing comprises a lancet disposed adjacent to the sample inlet.

8. A system according to claim 6, in which the sample conduit (47) is selected from a capillary conduit and a microfluidic conduit.

9. A system according to claim 6, in which the housing comprises an overflow chamber (48) fluidically connected to a distal side of the analysis chamber and a viewing window (49A) configured to allow a user visually detect the presence of whole blood in the overflow chamber.

10. A system according to claim 6, in which the housing comprises a venting conduit (49B) to vent air from the analysis chamber.

11. A system according to claim 1, in which the RF transmitter is configured to generate a resonant RF input signal with a frequency range of 0.5 to less than 20 kHz.

12. A system according to claim 1, in which the computing device comprises:
an algorithm trained using complex impedance training data obtained from blood samples from a training set of subjects and corresponding known specific cell type enumeration;
a processor configured to:
receive a determined complex impedance value for a blood sample from a test subject; and
apply the algorithm to the determined complex impedance value to calculate a cell count of the specific cell type in the of the blood sample from the test subject.

13. A system according to claim 12, in which the algorithm is a Support Vector Machine (SVM) machine learning algorithm.

14. A method of enumerating a specific cell type in a whole blood sample employing the system of claim 1, the method comprising the steps of
adding a whole blood sample to the analysis chamber of the cartridge;
coupling the cartridge to the reader device;
actuating the RF transmitter to provide an input RF signal to the impedance microsensor;
receiving by the RF receiver an output RF signal from the impedance microsensor in response to the input RF signal;
determining by the computing device a complex impedance value of the whole blood sample based on the output RF signal;
analysing by the computing device the complex impedance value of the whole blood sample to calculate a cell count of the specific cell type in the whole blood sample;
optionally, displaying the calculated cell count a graphical display of the reader device; and
detaching the cartridge form the reader device, wherein the whole blood sample that is analysed is untreated.

15. A method according to claim 14, which is a method of determining a neutrophil count of the whole blood sample.

16. A method according to claim 14, in which the whole blood sample is not treated to lyse erythrocytes.

17. A method according to claim 14, in which a blood cell binding agent is not added to the whole blood sample.
